(19) United States
(12) Patent Application Publication
(10) Pub. No.: US 2015/0157568 A1
(43) Pub. Date: Jun. 11, 2015

(54) Treatment of Spasticity with Intrathecal Dantrolene

(71) Applicant: Allergan, Inc., Irvine, CA (US)

(72) Inventor: Cun-Jian Dong, Irvine, CA (US)

(21) Appl. No.: 14/560,662

(22) Filed: Dec. 4, 2014

Related U.S. Application Data
(60) Provisional application No. 61/912,120, filed on Dec. 5, 2013.

(51) Int. Cl. A61K 9/00; A61K 31/4178

(52) U.S. Cl. CPC A61K 9/0085; A61K 31/4178

(57) ABSTRACT

The present invention is directed to a method of treating spasticity in a human subject, comprising administering intrathecally to the subject a therapeutically effective amount of dantrolene or a pharmaceutically acceptable salt thereof. The administration can be through a chronical drug delivery device such as an intrathecal pump.

### CROSS REFERENCE TO RELATED APPLICATIONS

This patent application claims priority to and the benefit of U.S. Provisional Patent Application No. 61/912,120, filed Dec. 5, 2013, the disclosure of which is hereby incorporated by reference in its entirety.

### FIELD OF THE INVENTION

The present invention is directed to a method of treating spasticity patients with intrathecally applied dantrolene.

### BACKGROUND OF THE INVENTION

Spasticity is a common secondary disabling condition following many neurological disorders such as stroke, cerebral palsy, spinal cord injury, and multiple sclerosis. It is characterized by increased muscle tone (hypertonus), increased involuntary somatic reflexes (hyperreflexia), clonus, and painful muscle spasms and increased resistance in response to stretch [Kheder & Nair, 2012]. Drug therapy for spasticity is symptomatic with the aim of increasing functional capacity and relieving discomfort.

Dantrolene is an FDA approved oral drug for the treatment of spasticity [Kita & Goodkin, 2000]. Systemically administrated dantrolene, especially at high doses, effectively reduces muscle stiffness and pain and improves the quality of life in many patients [Pinder et al., 1977; Ketel & Kolb, 1984], but unfortunately the risk of some serious side effects, such as fetal hepatotoxicity [Chan, 1990; Strommen, 2013] and generalized muscle weakness [Verrotti et al., 2006], also increases significantly as the dose increases, particularly at doses above 400 mg/day [Dantrolene FDA approved label], which often limits clinical utility of dantrolene [Strommen, 2013].

Over the last decade, availability of new chronic spasticity animal models [Bennett et al., 2004; Marsala et al., 2005] has significantly facilitated understanding the underlying pathophysiology of spasticity and the mechanism of action of antispasticity drugs [Murray et al., 2010], as well as testing the efficacy of new therapies and novel drug candidates [Kakinohana et al., 2012].

While the therapeutic efficacy of dantrolene in spasticity is generally believed to be produced by its direct action on the contractile mechanism of skeletal muscle to decrease the force of contraction [Pinder et al., 1977; Kheder & Nair, 2012; see also the “Clinical Pharmacology” section in Dantrolene FDA approved label ], additional sites of action of dantrolene have not been thoroughly explored and tested in appropriate animal models. Compared with systemic (such as oral or intravenous) drug administration, local drug delivery (such as intrathecal, as in the case of baclofen for the treatment of spasticity [Kita & Goodkin, 2000; Kheder & Nair, 2012]) at site of action can often improve the efficacy and substantially reduce the amount of drug needed which in general can significantly reduce systemic side effects.

EP2548594 refers to intrathecal baclofen pharmaceutical dosage forms and related delivery system.

US20120040970 refers to a method for rapidly and reliably delivering dantrolene, or derivatives thereof, alone or in combination with other compounds, to the systemic circulation by administration via the nasal route to produce rapid onset of beneficial effects in the treatment or prevention of malignant hyperthermia (MH), spasticity, and Ecstasy intoxication.

US20070065463 refers to topical formulations and methods of treating a migraines and/or cluster headaches, muscle sprains, muscle spasms, spasticity, tension headaches, tension related migraines and related conditions associated with muscle tension and pain with a therapeutically effective amount of an ergot alkaloid, skeletal muscle relaxant (e.g., dantrolene), serotonin agonist, and combinations thereof.

### BIBLIOGRAPHY

Chan C H. (1990) Dantrolene sodium and hepatic injury. Neurology, 40:1423-7.

Bennett D J, Sanelli L, Cooke C L, Harvey P J, Gorassini M A. (2004) Spastic long-lasting reflexes in the awake rat after sacral spinal cord injury. J Neurophysiol, 91: 2247-2258.

Dantrolene FDA approved label:

http://dailymed.nlm.nih.gov/dailymed/lookup.cfm?setid=c3c45b274786-456e-95d6-2e38ef36190a

Ketel W B, Kolb M E. (1984) Long-term treatment with dantrolene sodium of stroke patients with spasticity limiting the return of function. Curr Med Res Opin, 9:16116-9.

Kheder A, Nair K P. (2012) Spasticity: pathophysiology, evaluation and management. Pract Neurol. 2012 October; 12(5):289-98.

Kita M, Goodkin D E. (2000) Drugs used to treat spasticity. Drugs, 59:487-495.

Marsala M, Hefferan M P, Kakinohana O, Nakamura S, Marsala J, Tomori Z. (2005) Measurement of peripheral muscle resistance in rats with chronic ischemia-induced paraplegia or morphine-induced rigidity using a semi-automated computer-controlled muscle resistance meter. J Neurotrauma, 22:1348-1361.

Kakinohana O et al., (2012) Combinational spinal GAD65 gene delivery and systemic GABA-mimetic treatment for modulation of spasticity. PLoS One, 7(1):e30561.

Murray K C et al., (2010) Recovery of motoneuron and locomotor function after spinal cord injury depends on constitutive activity in 5-HT2_{c} receptors. Nat Med, 16:694-700.

Pinder R M, Brogden R N, Speight T M, Avery G S. (1977) Dantrolene sodium: a review of its pharmacological properties and therapeutic efficacy in spasticity. Drugs, 13:3-23.

Strommen J A. (2013) Management of spasticity from spinal cord dysfunction. Neurol Clin, 31:269-286.

Verrotti A, Greco R, Spalice A, Chiarelli F, Iannetti P (2006) Pharmacotherapy of spasticity in children with cerebral palsy. Pediatr Neurol, 34:1-6.

### SUMMARY OF THE INVENTION

The present invention provides a method of treating spasticity patients with an intrathecally applied, therapeutically effective amount of dantrolene (dantrolene in different forms, solution, polymer etc).

### BRIEF DESCRIPTION OF THE DRAWINGS

FIG. 1 shows the structure of dantrolene sodium.

FIG. 2 describes the rat spasticity model. (A,B) Electromyogram (EMG) responses (recorded from the gastrocnemius muscle) from an awake, but restrained normal and representative spastic rat when the ankle is rotated at velocity of (40°/3 sec) using a computer-controlled ankle rotational device. The presence of spasticity is identified by the appearance of burst EMG activity (active EMG). (C) To calculate the percent of inhibition of spastic muscle activity by a test agent during ankle dorsiflexion, animals were anaesthetized with isoflurane at the end of the experiment and the magnitude of active EMG during ankle dorsiflexion measured under isoflurane anaesthesia is then used as the maximum possible effect and is defined in each spastic animal. All drug treatment data generated after treatment are then normalized in using the maximum effect seen under isoflurane anaesthesia.

FIG. 3 shows that systemic administration (intraperitoneal (IP) injection) of dantrolene reduces spastic activity in the awake spastic rats in a dose dependent manner. At the highest dose tested (50 mg/kg, single injection), the spastic activity is reduced to about 30% of the pre-treatment level (70 % reduction) for at least 2 hours. These results confirm the efficacy of systemic dantrolene in this spastic rat model, which is also similar to that observed in human spasticity patients.

FIG. 4 shows that intrathecally (IT) administrated dantrolene (a single 250 μg bolus injection) has a potent anti-spastic effect, which is at least as robust as, if not more than, that with the highest (50 mg/kg) systemic administrated dantrolene in the awake spastic rats (n=6).

FIG. 5 shows representative electromyograms (red traces) recorded at different times following IT dantrolene injection from 3 of those rats. Isoflurane anesthesia that virtually eliminated all spastic activity in this rat model is used at the end of the experiment as the positive control for 0% (maximal anti-spastic effect).

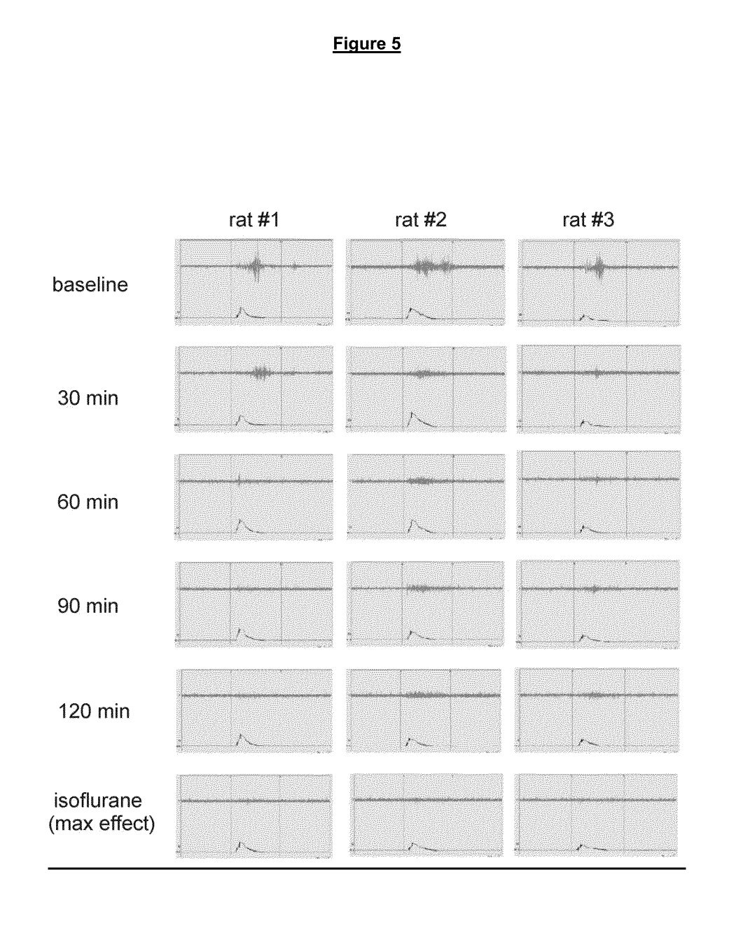

### DETAILED DESCRIPTION OF THE INVENTION

### Embodiments of the Invention

The present invention provides a method of treating spasticity in a human subject comprising administering intrathecally to said subject in need of such treatment a therapeutically effective amount of dantrolene or a pharmaceutically acceptable salt thereof.

As used herein, the terms “intrathecal (IT) administration,” “intrathecal injection,” “intrathecal delivery,” or grammatic equivalents, refer to an injection anywhere into the spinal canal (intrathecal space surrounding the spinal cord). In some embodiments, “intrathecal administration” or “ intrathecal delivery” according to the present invention refers to IT administration or delivery via the thoracic, lumbar, and sacral segments/regions of the spinal cord.

Salts of dantrolene include pharmaceutically acceptable base addition salts, pharmaceutically acceptable metal salts, ammonium, and alkylated ammonium salts. Examples of metal salts include lithium, sodium, potassium, magnesium salts and the like. Base salts include, but are not limited to, those formed with pharmaceutically acceptable cations, such as sodium, potassium, lithium, calcium, magnesium, ammonium and alkylammonium. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

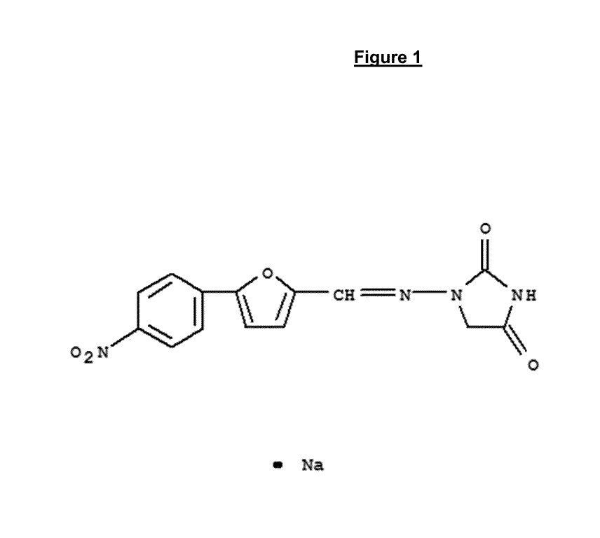

Examples of Quaternary ammonium salts of dantrolene have been disclosed in the publication “Synthesis and skeletal muscle relaxant activity of quaternary ammonium salts of dantrolene and clodanolene”, K. O. Ellis, R. L. Whatie Jr., G. C. Wright, F. L. Wessels, J. Pharm Sci., Volume 69, Issue 3, pages 327-331, March 1980. In one embodiment, the pharmaceutically acceptable salt is the sodium salt, i.e., the compound has the formula as set forth in FIG. 1.

Intrathecal delivery of dantrolene can be done by either a bolus injection or a continuous infusion. A bolus injection is defined as the injection of a drug (or drugs) in a relatively large quantity (called a bolus) at once, which is the opposite of gradual administration (e.g., intravenous infusion). Continuous infusion is defined as the administration of a drug or drug combination over a prolonged period of time (chronical administration).

In one embodiment, the dantrolene may be administered chronically, i.e., by continuous intrathecal infusion.

In another embodiment, the administration by continuous intrathecal infusion is carried out using an implanted pump.

In another embodiment, the administration of dantrolene may potentially result in reduction or elimination of side effects from systemic administration of dantrolene, such as liver toxicity and generalized muscle weakness.

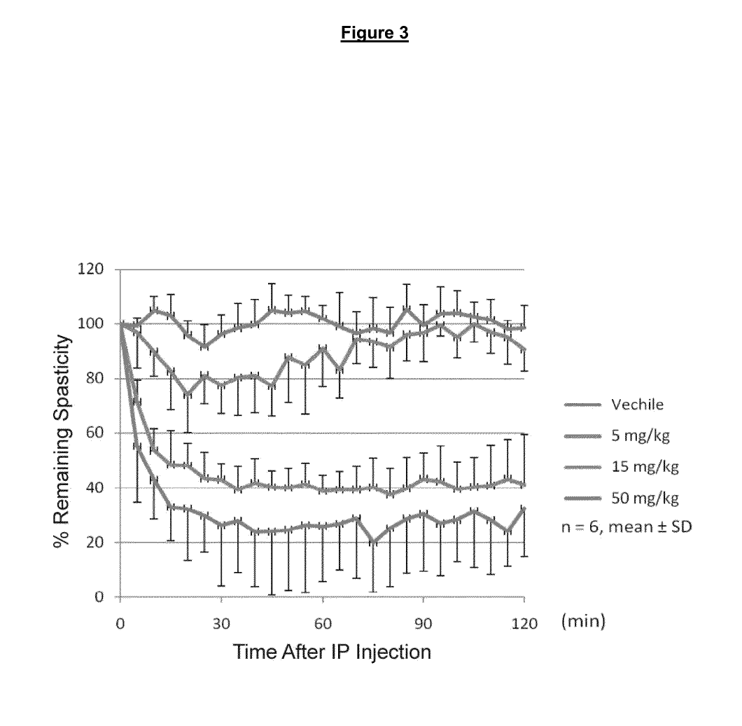

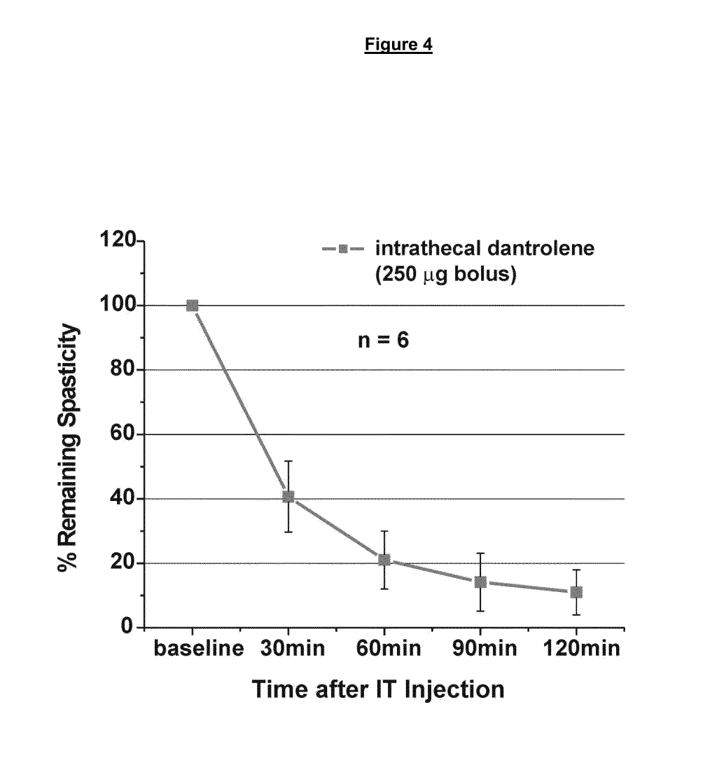

In another embodiment, the administration of dantrolene may potentially result in a better efficacy (comparing results in FIGS. 3 and 4).

In another embodiment, the dantrolene is administered intrathecally at a dose of about 0.05 mg/kg per day to about 6 mg/kg per day. In another embodiment, this does is administered with an intrathecal infusion pump.

In another embodiment, the dantrolene is administered as a single bolus dose intrathecal injection.

### Assays

### Spastic Rat Model

Animal: Male Sprague-Dawley rats (approximately 350 gram body weight) were used in the assay. The rats were housed at room temperature (65-82° F.) and relative humidity within the range between 30 to 70%. The room will be illuminated with fluorescent lighting on a daily 12 hour light/dark cycle. All animals will have free access to dry food. Municipal water will be freely available.

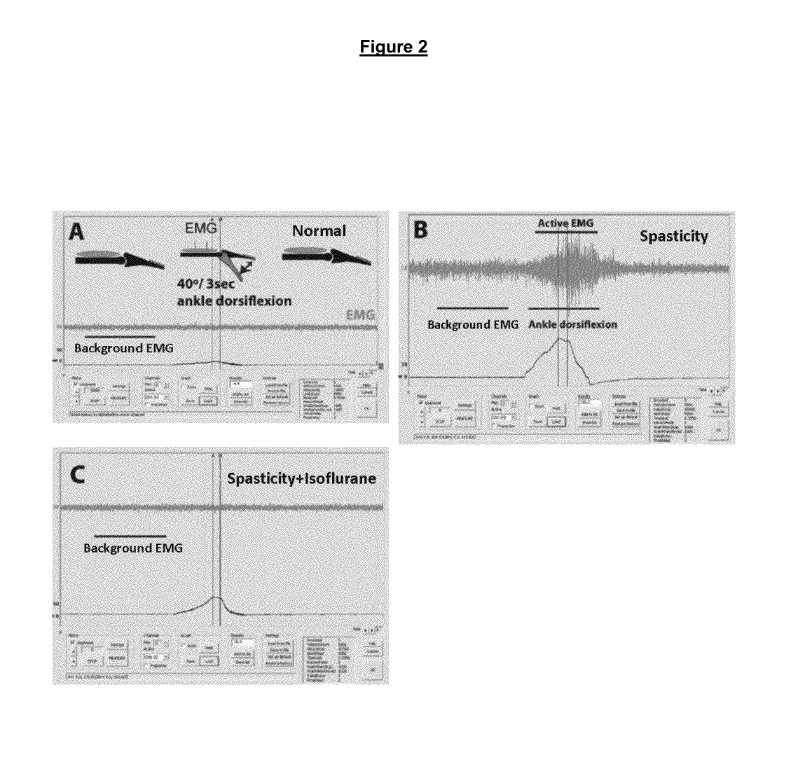

Spasticity development and measurement: Rats were exposed to transient spinal cord ischemia by aortic balloon occlusion for 10-12 minutes. Animals with fully developed spasticity will be selected at 4-8 weeks after ischemic injury and prepared for spasticity measurement. Presence of spasticity will be defined by increased peripheral muscle resistance measured during computer-controlled ankle dorsiflexion and correlative changes in EMG (electromyography) activity measured in gastrocnemius muscle (see FIG. 2).

An increased EMG activity induced by ankle dorsiflexion is a consistent and reliable measure of spasticity. Before administration of dantrolene, baseline EMG measurements were conducted. Animals with identified spasticity were then assigned to the experimental groups to be treated with dantrolene or vehicle through intraperitoneal and intrathecal routes.

Intraperitoneal dosing: Three different doses (5, 15, 50 mg/kg) of dantrolene and vehicle were injected intraperitoneally (6 rats per treatment/dose). After dantrolene administration, correlative changes in EMG were recorded at 15, 30, 60, 90, and 120 minutes following intraperitoneal injection. Changes in baseline EMG activity and ankle-rotation evoked EMG activity were then analyzed and the efficacy of treatment on spasticity calculated.

Intrathecal dosing: Animals with identified spasticity were implanted intrathecally with a fine plastic (e.g., PE-10) catheter for bolus drug delivery. Two to three days after intrathecal catheter implantation, the baseline spasticity response was measured and animals were then injected IT with dantrolene (250 μg, 6 rats) and spasticity response was measured for 2 hrs. In control animals (6 rats) vehicle was injected only.

Each and every reference disclosed herein, whether a patent publication/granted patent or a scientific publication is incorporated by reference herein for all purposes.

It is to be understood that the foregoing describes preferred embodiments of the present invention and that modifications made be made therein without departing from the scope of the present invention as set forth in the claims.

What is claimed is:

1. A method of treating spasticity in a human subject comprising administering intrathecally to said subject in need of such treatment a therapeutically effective amount of dantrolene or a pharmaceutically acceptable salt thereof.

2. The method of claim 1, wherein the pharmaceutically acceptable salt is the sodium salt.

3. The method of claim 1, wherein the dantrolene is administered by continuous infusion.

4. The method of claim 3, wherein the infusion is by an implanted pump.

5. The method of claim 1, wherein the administration results in reduction or elimination of damaging side-effects from systemic administration of dantrolene.

6. The method of claim 5, wherein the side effects are selected from the group consisting of fetal liver toxicity and generalized muscle weakness.

7. The method of claim 1, wherein the dantrolene is administered at a dose of about 0.05 mg/kg per day to about 6 mg/kg per day.

8. The method of claim 1, wherein the dantrolene is administered as a single bolus injection.
